(19) United States
(12) Patent Application Publication
(10) Pub. No.: US 2015/0216418 A1
(43) Pub. Date: Aug. 6, 2015

(54) INSPECTION OF DENTAL ROOTS AND THE ENDODONTIC CAVITY SPACE THEREIN

(71) Applicant: DENTSPLY International Inc., York, PA (US)

(72) Inventors: DAN AMMON, TULSA, OK (US); STEPHEN RIGSBY, SAND SPRINGS, OK (US); WILKINSON KEVIN, BIXBY, OK (US); BRIAN ATKINSON, Fishers, IN (US)

(21) Appl. No.: 14/616,650

(22) Filed: Feb. 6, 2015

Related U.S. Application Data
(60) Provisional application No. 61/936,513, filed on Feb. 6, 2014.
(60) Provisional application No. 62/049,624, filed on Sep. 12, 2014.

(51) Int. Cl. A61B 5/00; A61B 1/24

(52) U.S. Cl. CPC A61B 5/0088; A61B 1/24; A61B 5/0071; A61B 5/742

(57) ABSTRACT

A system and method for acquiring and sharing in real time video and/or still images with a tooth or tooth root during a dental procedure using an endoscope.

### CROSS-REFERENCE TO RELATED APPLICATIONS

This patent application claims the benefit of and priority to U.S. Provisional Application Ser. No. 61/936,513, filed on Feb. 6, 2014 and U.S. Provisional Application Ser. No. 62/049,624, filed Sep. 12, 2014, which are herein incorporated by reference for all purposes.

### FIELD OF INVENTION

The present invention relates to the use of a dental micro-endoscope combined with an imaging camera, multiple optical fibers for coupling illumination and excitation energy, a wired or wireless transmission system for sending the still image or video information to a heads-up display of the dental root canals and other intra-oral areas in real time to patients, practitioners, consultants, and archives. In addition, dyes and fluorphores for staining and tagging various root canal native materials well as foreign material can be used with different excitation energies than in current dental scopes to fluoresce and visualize these materials; these images can be qualitatively and quantitatively evaluated by algorithms in a computer to provide mapping of the canal and material for comparative evaluation or diagnostics. Additional diagnostic capabilities of the canal walls and dentin by use of optical coherence tomography are also possible by use of different optical tips and computer algorithm analysis.

### BACKGROUND OF THE INVENTION

Endodontic microscopes can be used for observing, cleaning and enlarging the endodontic cavity space (“ECS”), also known as the root canal system of a human tooth. The unprepared root canal is usually a narrow channel that runs through the central portion of the root of the tooth. Cleaning and enlargement of the ECS can be necessitated by the death or necrosis of the dental pulp, which is the tissue that occupies that space in a healthy tooth. This tissue can degenerate for a multitude of reasons, which include tooth decay, deep dental restorations, complete and incomplete dental fractures, and traumatic injuries or spontaneous necrosis due to the calcification and ischemia of the tissue, which usually accompanies the ageing process. Similar to a necrotic or gangrenous appendix, the complete removal of this tissue is paramount, if not urgent, because of the subsequent development of infections or dental abscesses, septicemia, and even death.

The root canal system of a human tooth is often narrow, curved and calcified, and can be extremely difficult to negotiate or clean. Indeed, the conventional microscopes currently available are frequently inadequate in the complete observation of removal of the pulp and the efficient enlargement of the ECS. Furthermore, they are usually predisposed to one observing angle at a time, further slowing a clinical procedure. Often a mirror must be used for observation angles that cannot be seen by the standard dental microscopes and that those views are limited to the top portion of the canal for complex or curved canals.

Broken instruments are usually difficult, if not impossible to remove, often necessitating the removal of the root or tooth. Injury to the tooth, which occurs as the result of a frank perforation or alteration of the natural anatomy of the ECS, can also lead to failure of the root canal and tooth loss. Broken files are often out of the focal points of the dental microscope.

The unprepared root canal of the tooth usually begins as a narrow and relatively parallel channel. The portal of entry or the orifice and the portal of exit or foramen are relatively equal in diameter. To accommodate complete cleaning and filling of the canal and to prevent further infection, the canal must usually be prepared. The endodontic cavity preparation (“ECP”) generally includes progressively enlarging the orifice and the body of the canal, while leaving the foramen relatively small. The result is usually a continuous cone-shaped preparation. While the clinical procedure is often done via a dental microscope, typically only the pulp chamber and canal opening are observed by the microscope. Viewing the canals themselves is difficult because of the root canal complex anatomy.

Video dentistry was described in the 1980s but not widely adopted until decades later. The dental microscope often has a microscope with an articulating arm and software that allows for digital images and wired display to a monitor. A dental microscope can be used during routine teeth inspection. However, the technique is limited because viewing angles between teeth is nearly impossible depending upon the anatomy. This limitation can be overcome with the use of fiber optics.

Wearable glasses for viewing simulated three-dimensional images on a television screen or monitor have been described since the 1990s and are commonplace today. 3D viewing glasses have also been coupled to video gaming consoles. The wearable heads-up display concept was reduced to practice early in this millennium. A comprehensive description of one embodiment known as Google Glass was recently described (United States Patent Application 20130044042, which is herein incorporated by reference into this disclosure for all purposes). While the inventors of the display envision numerous applications within the computer industry, Google Inc. has made prototype glasses available to a limited number of beta testers to promote additional heads-up display applications in other industries.

While dentists currently use a dental microscope to view images of teeth during examination, consultation with specialists normally requires that still images be sent to the consultant, delaying completion of the case. The heads-up display allows for ready real-time viewing of still or video images of teeth during a case, with any or all involved parties able to view simultaneously.

Various detection devices for teeth, wearable devices, and systems for displaying three-dimensional images are described in U.S. Pat. No. 6,584,341 issued to Mandelis et al. for “Method and apparatus for detection of defects in teeth;” U.S. Pat. No. 4,468,197 issued to Provost for “Apparatus and method for detecting cavities;” U.S. Pat. No. 8,542,326 issued to MacNaughton et al. for “3D shutter glasses for use with LCD displays;” US Patent Application 20130044042 by Olsson; Maj Isabelle et al. for “Wearable device with input and output structures;” U.S. Pat. No. 6,132,211 issued to Peithman for “Portable dental camera, system and method;” U.S. Pat. No. 5,836,762 issued to Peithman for “Portable dental camera system and method;” U.S. Pat. No. 5,487,661 issued to Peithman for “Portable dental camera and system;” US Patent Application 20050225630 by Childers et al for “Method and system for displaying an image in three dimensions” and U.S. Pat. No. 5,682,196 issued to Freeman for “Three-dimensional (3D) video presentation system providing interactive 3D presentation with personalized audio responses for multiple viewers.” The entire disclosures of these patents and applications are herein incorporated by reference into this disclosure for all purposes.

Endodondists currently use radiographs or computed tomography machines to evaluate the shape and condition of the root canal system and tooth to plan their preparation and treatment. The canal is then prepared by shaping, irrigating, and energizing various disinfecting and cleaning fluids with different types of energies (i.e. sonic, ultrasonic, photon-induced photoacoustic streaming). The current methods of determining the level of cleaning the canal has reached in an operational clinical setting are inadequate; inadequate cleaning is thought to be a leading cause of RCT failure and results either in retreatment or more serious dental procedures. A device and method that is capable of providing the endodontist information on the degree of preparation and cleaning that has been achieved is key in improving the standard of care.

Besides, the normal visualization by visible light described in the above paragraphs, native material in the root canal such as collagen (pulp), can be highlighted via fluorescence by using excitation energy in the UV spectrum. In addition, bacteria, mold and other undesirable matter or states of the native tooth can also be highlighted via fluorescence by use of the appropriate tagging fluorophores or staining dyes. This invention envisions all of the above and various derivatives in providing a system that provide qualitative assessments and quantitative diagnostics of the root canal as well as other intra-oral areas of the tooth and mouth.

Examples of related technology include US patent application 20130044042 for Wearable device with input and output structures (e.g. Google Glass); U.S. Pat. No. 6,584,341 for a Method and apparatus for detection of defects in teeth (e.g., Uses laser induced frequency doman infrared photothermal radiometry and AC modulated luminescence signals to detect defects and caries in teeth intra-orally); U.S. Pat. No. 4,468,197 for an apparatus and method for detecting cavities (e.g., shines an incandescent light through a tooth using fiber optics. Signal is picked up on the other side of the tooth with a coherent fiber optic bundle and that image is displayed on a screen. Used for detecting caries with normal eyesight technique); U.S. Pat. No. 8,542,326 for 3D shutter glasses for use with LCD displays (e.g., a viewing system for viewing video displays so that they appear to be 3D); U.S. Pat. No. 6,132,211 for a portable dental camera, system and method (e.g., a dental camera system with additional power sources as well as sterilizing or protective jackets); U.S. Pat. No. 5,836,762 for a portable dental camera system and method (e.g., a dental camera system including a method of sterile use by putting a disposable sleeve over the handpiece for each patient or removable jacket over the handpiece that can be autoclave sterilized between patient use); U.S. Pat. No. 5,487,661 for a portable dental camera and system (e.g., a camera in a handpiece with an elongated cylindrical body wall and a rigid cover which is sterilizable); US Patent application 20050225630 A1 for a method and system for displaying an image in three dimensions (e.g., a method to display 3D images with a single light engine by using different colors in the left frame than the right frame); U.S. Pat. No. 5,682,196 for a three-dimensional (3D) video presentation system providing interactive 3D presentation with personalized audio responses for multiple viewers (e.g., a system allows detection of a choice (i.e. amongst several 3D options in front of him) made by the user and then provides individualized responses based on that choice: Interactive realism); CN Patent 2617308Y for a digital optical fibre root canal microendoscope device (e.g., a mini-endoscope with visible light); Paper in European Journal of Medical Research, Eur J Med Res (2006) 11: 123-127 for the development of a New Micro-Endoscope for Odontological Application (e.g., a mini-endoscope for use in various dental disciplines using visible light including endodontoloy. Includes lab results. The light source was a 250 W halogen lamp); Article in Alpha Omegan 104:1/2, 2011 for endoscopy in endodontics (e.g., a mini-endoscope for use in various dental disciplines using visible light including endodontoloy); U.S. Pat. No. 4,646,722 for a protective endoscope sheath and method of installing same (e.g., an endoscope sheath having a flexible tube surrounding the elongated core of an endoscope. The flexible tube has a transparent window near its distal end positioned in front of the viewing window of the endoscope); U.S. Pat. No. 4,825,850 for contamination protection system for endoscope control handles (e.g., a contamination control system for endoscopes having a handle, an insertion tube projecting from the handle and control knobs projecting from the handle); U.S. Pat. No. 4,852,551 for contamination-free endoscope valves for use with a disposable endoscope sheath (e.g., an endoscope and valve system specially adapted for use with a disposable sheath having multiple channels and tubes extending); U.S. Pat. No. 4,907,395 for packaging system for disposable endoscope sheaths (e.g., Packaging system for disposable sheaths to keep them non-contaminated during shipment as well as when one is pulled out from the package); U.S. Pat. No. 4,947,827 for a flexible endoscope; U.S. Pat. No. 5,025,778 for an endoscope with potential channels and method of using the same; U.S. Pat. No. 5,193,525 for an antiglare tip in a sheath for an endoscope; U.S. Pat. No. 5,271,381 for a vertebrae for a bending section of an endoscope; U.S. Pat. No. 5,329,887 for an endoscope control assembly with removable control knob/brake assembly; U.S. Pat. No. 5,337,734 for a disposable sheath with optically transparent window formed continuously integral therewith; U.S. Pat. No. 5,443,781 for a method of preparing disposable sheath with optically transparent windows formed continuously integral therewith; U.S. Pat. No. 5,447,148 for an endoscopic contamination protection system to facilitate cleaning of endoscopes; U.S. Pat. No. 5,483,951 for working channels for a disposable sheath for an endoscope; U.S. Pat. No. 5,518,501 for an endoscopic contamination protection system to facilitate cleaning of endoscopes; U.S. Pat. No. 5,520,607 for a holding tray and clamp assembly for an endoscopic sheath; U.S. Pat. No. 5,626,553 for an endoscope articulation system to reduce effort during articulation of an endoscope; U.S. Pat. No. 5,667,476 for an endoscope articulation system to reduce effort during articulation of an endoscope; U.S. Pat. No. 5,685,822 for an endoscope with sheath retaining device; U.S. Pat. No. 5,692,729 for a pressure equalized flow control apparatus and method for endoscope channels; U.S. Pat. No. 5,702,348 for a disposable endoscopic sheath support and positioning assembly; U.S. Pat. No. 5,827,177 for an endoscope sheath assembly with isolating fabric sleeve; U.S. Pat. No. 5,876,329 for an endoscope with sheath retaining device; U.S. Pat. No. 6,174,280 for a sheath for protecting and altering the bending characteristics of a flexible endoscope; U.S. Pat. No. 6,190,330 for an endoscopic location and vacuum assembly and method; U.S. Pat. No. 6,350,231 for an apparatus and method for forming thin-walled elastic components from an elastomeric material; U.S. Pat. No. 6,461,294 for an inflatable member for an endoscope sheath; U.S. Pat. No. 6,530,881 for a sheath apparatus for endoscopes and methods for forming same; U.S. Pat. No. 6,579,582 for an apparatus and method for forming complex-shaped components in a heated polymeric film; which are herein incorporated by reference for all purposes.

Current Technology Limitations

When the clinician can see something that is challenging, abnormal, or beyond his/her capabilities of treatment, the current practice is to refer the patient to an expert or to send the pictures to a higher skill practitioner for consultation. The patient then returns at a different time to the original clinician or goes to the expert to receive the treatment. This may cause a significant delay in treatment as well as reduced customer satisfaction.

The small size of the canal results in an image fiber with a small number of pixels, 3,000 pixels for ˜0.40 mm OD tip, ˜6,000 pixels for a ˜0.60 mm tip, and 10,000 pixels for ˜1.0 mm tip. The latter two are limited in use mainly for the top ⅓ of the canal or at the top of the canal due to the size. The smaller size ˜0.4 mm can go into the canal further but has less resolution. This limits the image quality and the capability of discerning the cleanliness of the canal, objects or material in the canal, or canal defects or morphology. At this time, there appears to be no product that has been accepted by the market for normal use. As mentioned previously, there is literature describing the current technology, but it is not in normal use due to the above limitations.

Endodondists currently use radiographs or computed tomography machines to evaluate the shape and condition of the root canal system and tooth to plan their preparation and treatment. The canal is then prepared by shaping, irrigating, and energizing various disinfecting and cleaning fluids with different types of energies (i.e. sonic, ultrasonic, photon-induced photoacoustic streaming). The current methods of determining the level of cleaning the canal has reached in an operational clinical setting are inadequate; inadequate cleaning is thought to be a leading cause of RCT failure and results either in retreatment or more serious dental procedures. The devices described previously for root canal inspection use white light for normal vision inspection and do not allow detection of microbial infections such as bacteria or mold.

Hand-eye motion between the tip placement and watching the still images or video on the monitor is not ideal in that the clinician's focus is on a screen away from the patient's mouth.

In an attempt to overcome the limitations/problems of the prior art instruments, it is an object of this invention to provide one or more of the following improvements:

Improve usability by having the images and video from the mini-endoscope displayed on a heads-up display so the clinician can see inside the canal at the same time as he is looking inside the mouth and manipulating the optical tip and/or other instruments.

Improve usability by sending signals wirelessly from the handpiece 1) to the heads-up display, 2) to the clinicians IT network for storage, 3) to monitors for the patient or other support people in the office.

Improve clinical efficacy and efficiency by sending the signals wirelessly from the handpiece to the clinicians network and then to an external consultant, such as another endodontist, for real-time consultation and advice.

Improve the image quality and type by the addition of other illumination wavelengths such as IR and UV so that clinicians can determine, in the operational clinical setting, the level of preparation and cleaning he/has achieved in the canal as well as undesirable conditions of the tooth such as cracks. These additional wavelengths will fluoresce collagen (pulp) that remains and, with certain staining dyes and tagging fluorophores, molecules in the mouth and canal such as bacteria and molds.

Be capable of characterizing the root canal walls and dentin by employing optical coherent tomography (OCT) in a non-destructive manner by modifying the image fiber tip to include a fiber-optic splitter to create the sample reference arms of the interferometer for the data stream and OCT analysis algorithms in the handpiece or in a computer. This can also be used outside the tooth intra-orally.

### SUMMARY OF THE INVENTION

The present invention seeks to improve upon prior endoscopes by providing improved micro-endoscope system. In one aspect, the present invention provides a system for acquiring and sharing in real time video and/or still images with a tooth or tooth root during a dental procedure comprising an endoscope.

In another aspect, the present invention contemplates a method for acquiring and sharing in real time video and/or still images with a tooth or tooth root during a dental procedure comprising the step of illuminating a cavity of a root canal and acquiring still images and or video of the illuminated cavity of the root canal.

In yet another aspect, any of the aspects of the present invention may be further characterized by one or any combination of the following features: further comprising a heads up display in communication with the endoscope for displaying in real-time the video and/or still images wherein the endoscope includes a fiber optic material; wherein the fiber optic material is a quartz fiber so that other wavelengths including UV, IR, or a combination of both for excitation; wherein the endoscope further includes a removable sheath having a hydrophilic or hydrophobic exterior coating; wherein the endoscope employs an OCT design and software algorithms to image the root canal walls; further comprising a micro-fiberscope, an illumination source, a camera, a wired or wireless transmission system, and a display; wherein the display is a television, tablet, smartphone or computer monitor; wherein the display is a heads-up display; wherein the heads-up display is Google Glass; wherein the heads-up display is Samsung Galaxy Glass; wherein the fiber optic material is selected from the group consisting of polymeric optic fibers, glass-ceramic optic fibers, and quartz optic fibers; wherein the endoscope includes a fiberscope having a dental probe detachable from a main fiber optic cable; wherein the probe attaches to the main fiberoptic cable through a quick connector; wherein the probe is constructed of all polymeric materials; wherein the probe is resterilizable; wherein the probe is disposable; wherein the probe includes a probe tip that is modified or coated to make it hydrophilic; wherein the probe includes a probe tip that is modified or coated to make it hydrophobic; wherein the endoscope further includes a disposable sheath, which protects the probe from being biocontaminate; wherein the microfiber is 0.01 mm or larger; wherein the microfiber is between 0.1 mm and 10 mm; further comprising the step of applying fluorophores and/or dyes to a cavity with a root of a tooth to tag and/or stain bacteria, molds, and/or other molecules so they will fluoresce under UV, IR or other wavelengths for identification by the imaging system; wherein the applying fluorophores and/or dyes step includes using UV 270-370 nm to fluoresce collagen in the root canal; wherein the illumination step includes using blue visible light to fluorescence dentin in the root canal; wherein the video and/or still images are acquired using a micro-fiberscope, illumination source, and camera; wherein the video and/or still images are wirelessly transmitted to one or more heads-up displays; wherein the video and/or still images are transmitted via a wired connection to one or more heads-up displays or network with one or more monitors or heads-up displays; wherein the video and/or still images are transmitted over a wireless network to one or more heads-up displays; further comprising the step of transmitting images and/or videos with a patient, clinical staff, consultants for patient education, dental staff training, consultant referrals or consultations, or any combination thereof; wherein the video and/or still images of a feature or structure are acquired using a micro-fiberscope, fluorophore, illumination source of wavelength capable of effecting fluorescence of the fluorophore, and a camera; wherein the fluorophore is present naturally within the tooth; wherein the illumination source emits UV light of 270-370 nm wavelength; wherein the structure is collagen; wherein the fluorophore is sodium fluorescein; wherein the fluorophore is applied to the tooth after endodontic preparation from a solution; wherein the illumination source emits visible light; wherein the video and/or still images are transmitted to a display; wherein the display is selected from the group consisting of a television, a computer monitor, and a heads-up display; wherein the transmission is via a wireless signal; or any combination thereof.

It should be appreciated that the above referenced aspects and examples are non-limiting as others exist with the present invention, as shown and described herein. For example, any of the above mentioned aspects or features of the invention may be combined to form other unique configurations, as described herein, demonstrated in the drawings, or otherwise

### BRIEF DESCRIPTION OF THE DRAWINGS

The novel features of the invention are set forth with particularity in the appended claims. The invention itself, however, both as to organization and methods of operation, together with further objects and advantages thereof, may best be understood by reference to the following description, taken in conjunction with the accompanying drawings in which:

FIG. 1 is a perspective view of an endoscope handpiece assembly in accordance with one embodiment of the present invention;

FIG. 2 is a perspective view of a first treatment system, which includes the endoscope handpiece assembly shown in FIG. 1 in accordance with another embodiment of the present invention;

FIG. 3 is various top views of a root canal of a tooth that is being illuminated by an endoscope handpiece assembly of the present invention at various depths of the root canal;

FIG. 4 is a side view of a root canal of the tooth that is being illuminated at various depths shown in FIG. 3;

FIG. 5 is an exploded view another endoscope handpiece assembly in accordance with another embodiment of the present invention;

FIG. 6 is an exploded view another endoscope handpiece assembly in accordance with another embodiment of the present invention;

FIG. 7 is a cross-sectional side view of another end portion for an endoscopic handpiece assembly in accordance with another embodiment of the present invention;

FIG. 8 is a bottom view of the end portion shown in FIG. 7;

FIG. 9 is a zoomed in cross-sectional view of an intermediate portion of the end portion shown in FIG. 7;

FIG. 10 is a zoomed in cross-sectional view of a free end of the end portion shown in FIG. 7;

FIG. 11 is a perspective view of another end portion for an endoscopic handpiece assembly in accordance with another embodiment of the present invention;

FIG. 12 is a side view of the end portion shown in FIG. 11.

### DETAILED DESCRIPTION OF THE INVENTION

The present invention may provide an endoscopic handpiece assembly. More particularly, the present invention may provide a mini endoscopic handpiece assembly with a removable protective end portion (e.g., disposable guide tube) for illuminating and/or displaying and/or recording (either simultaneously, periodically, in combination, or separately) a root canal and/or cavity within a tooth or patient's oral cavity through a fiber optic cable or otherwise (e.g., micro-fiberscope attached to the body of the endoscopic handpiece assembly. It is appreciated that the endoscopic handpieces of the present invention may simultaneously illuminate and display inner cavity spaces of root canals during, in between, and/or after treatment of a root canal (e.g., cleaning and/or shaping of a root canal).

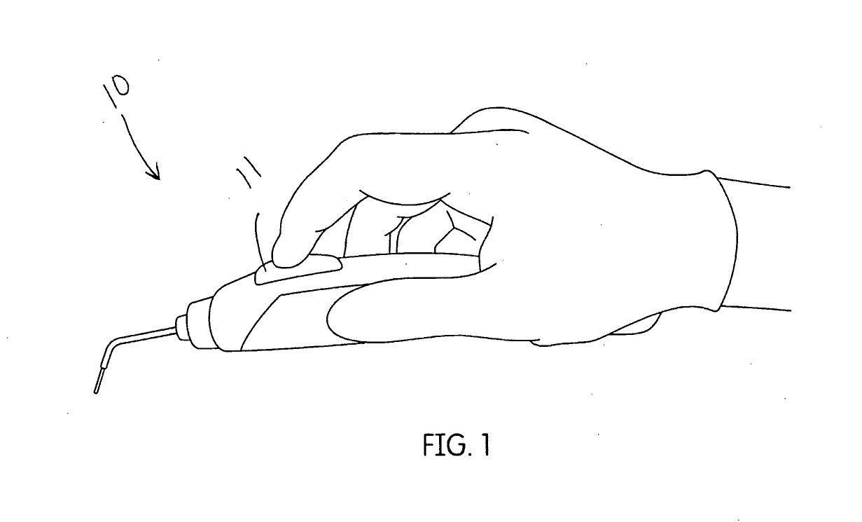

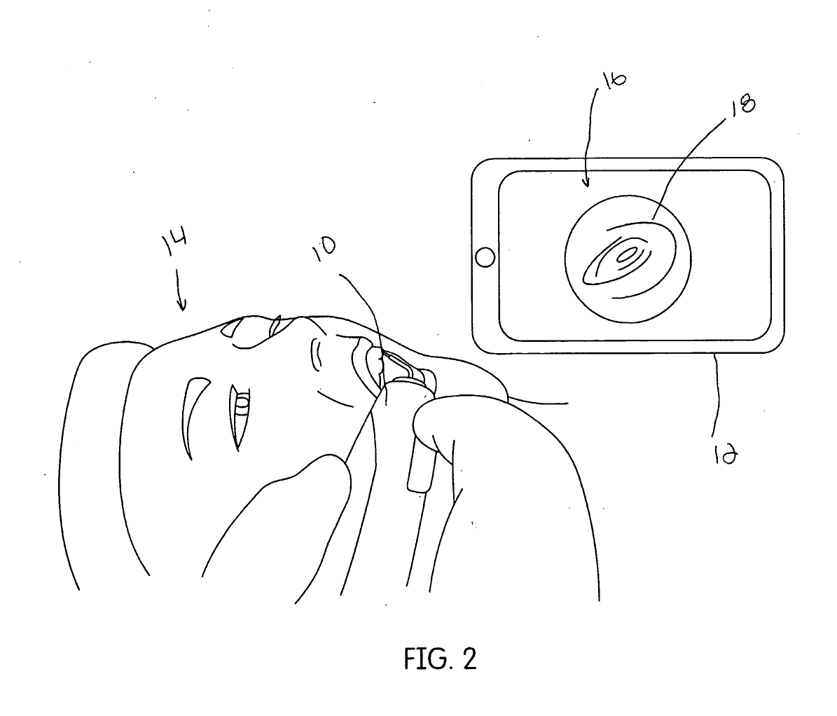

It is contemplated that in a live treatment case, the tooth of a patient undergoing an endodontic root canal treatment may be prepared using standard procedures whereby the infected pulp is excised before the canals are filed. To confirm adequate preparation, a mini-endoscope 10 having a power button 11 is advanced into the canals for inspection (FIGS. 1-2). A video stream of the illuminated canal is transmitted across a wireless (or wired) network to wearers of Google Glass (dentist, endodontist, or otherwise) or other heads-up display or otherwise display device 12 (e.g., monitor, tablet, laptop, or similar device and/or otherwise), which may be located in the same room with the treatment of the patient 14 and/or to a consultant/specialist or otherwise in a different location in real time or previously recorded.

Optionally, during a live treatment case, images 16 (e.g. still images and/or video streaming and/or recording) may be taken inside the tooth 18 at various times during the treatment procedure. The still images may be transmitted across a wireless (or wired) network to dental students, each wearing a pair of Google Glass (or other heads-up display) or display device, during an instructional session at their dental school. A GP or endodontist can also transmit streaming video, pictures or recorded video to a consultant or another dentist. The customer does not have to leave the chair to see another dentist for the procedure or come back later after the initial dentist gets a consultation.

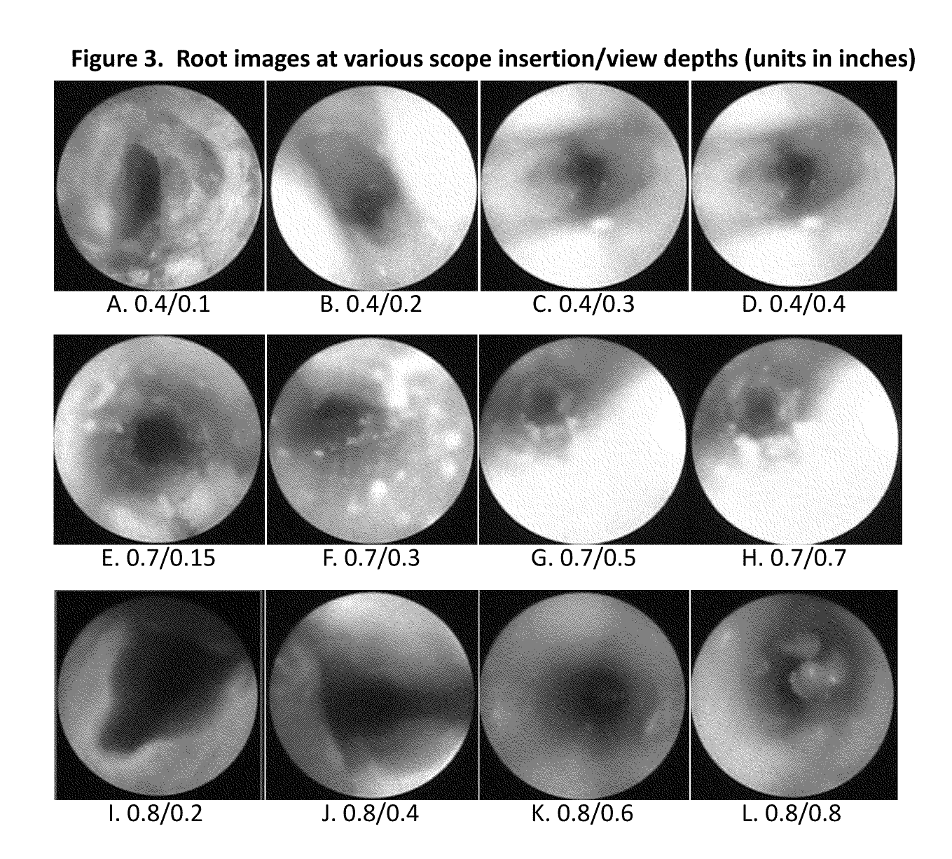

In one specific example, an endoscope embodiment is provided having a flexible shaft micro-fiberscope (Millescopell, Zibra Corp, Wesport Mass.) with the following specifications: Diameter=0.6 mm; 6,000 pixel image fiber; field of view (FOV)=65 degrees; AR-coated dual element objective lens; optimal focal length=5 mm; depth of focus (DOF)=3-12 mm; illumination source=6000 k white light LED; camera=½ format Sony EX sensor analog CCD. The ECS of a pulled human tooth was prepared by excising pulp from the roots using standard endodontic procedure. The ECS was then examined using the micro-fiberscope. Video images within the tooth and root canals were transmitted to the camera and recorded as the probe was advanced and retracted. Still images captured at total scope insertion depths of 0.4, 0.7 and 0.8 inches and viewing depths ranging from 0.1 to 0.8 inches as shown in FIG. 3. The video images were streamed in real time to a heads up display (Google Glass) and the still images transmitted later to both an external monitor and Google Glass.

The clinician can also use a larger endoscope, such as like 5 mm outer diameter, and heads up display to inspect teeth or other intra-oral areas with a patient. The clinician can also use the endoscope to show a customer or record a cavity in a heads up display.

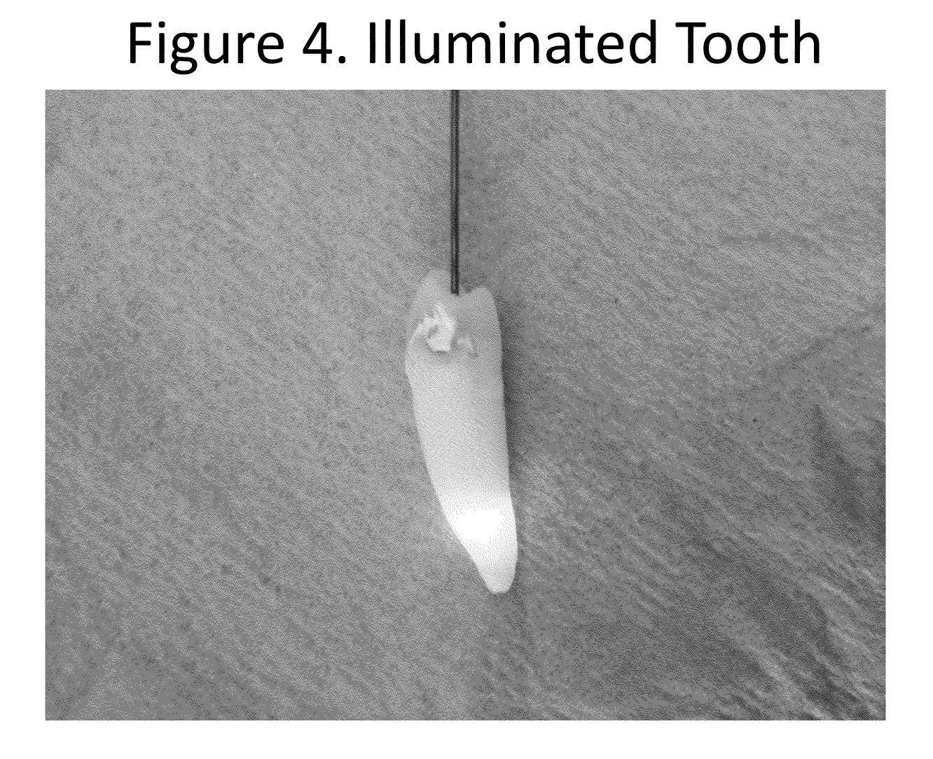

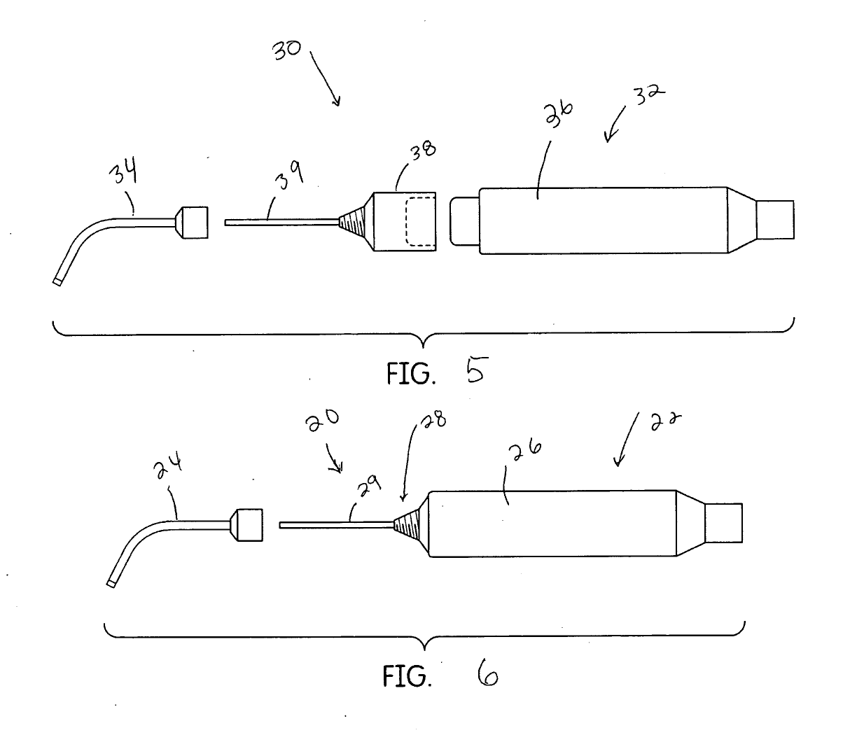

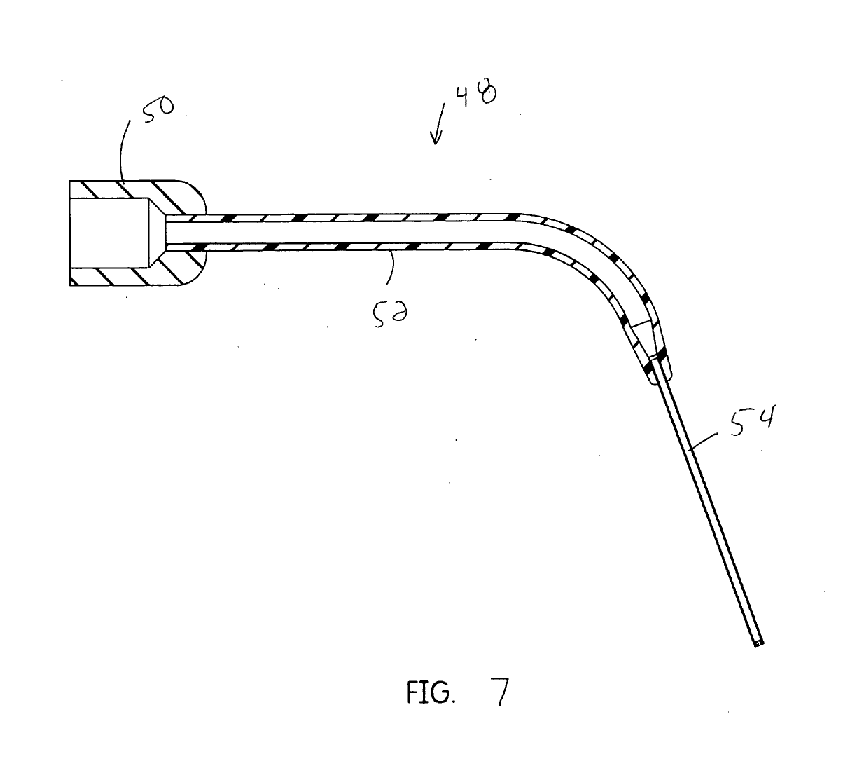

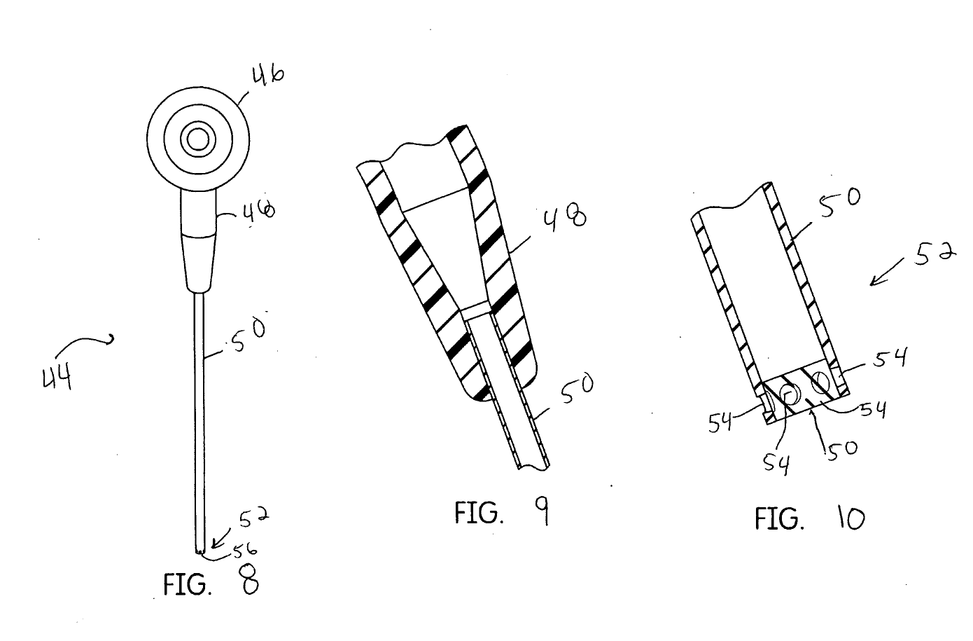

The tooth of patient undergoing endodontic root canal treatment is prepared using standard procedures whereby the infected pulp is excised before the canals are filled. To confirm adequate preparation, the canal can be filled with fluorescein for an appropriate amount of time. The micro-fiberscope described in the specific example above may include a light source emitting UV light over a wavelength range that spans 310 nm is advanced into the canals for inspection as shown in FIG. 4. In real-time with a patient, the video stream may be transmitted wirelessly from the handpiece to a practitioner and/or different endodontist or assistant wearing Google Glass or other heads-up display and/or a monitor in a different or same room.

The endodontist can evaluate the image for fluorescence indicative of caries and specifies additional excision or filling if residual caries is indicated or filling of the root space if not. The imagefiber can have an annulus of illumination optical fibers which are then encapsulated by a protective coating. The illumination fibers can be connected to one illumination source such as visible light.

In another embodiment has the annulus of illumination fibers in a mixture whereby more than one energy source, such as visible, IR, and UV, is connected to the different illumination fibers. In yet another embodiment, the illumination fibers may alternate around the annulus. In yet another embodiment, the illumination fibers may be provided in a different spacing pattern around the image fiber for different imaging or to change the handling characteristics of the flexible probe (e.g., removable end portion such as a disposable protective sheath). Another embodiment may include different materials of the illumination fibers that are utilized in the assembly. Fused quartz fibers may be used instead of normal glass fiber for improved optical and UV transmission properties. Polymeric optical fibers may be used instead of glass or quartz because they are typically more durable than glass and sometimes more cost effective. Materials including polymethyl methacrylate, polystyrene, and polycarbonate are appropriate are other alternative optical fiber materials. Fiber optic glass can also be treated to improve strength of each individual fiber. For example, ion-exchange treatment at the surface is used in the fabrication of Gorilla Glass (Corning Inc., Corning, N.Y.). In addition, the tips of the fiber-optic endoscopic probes (glass and plastic) can be treated or coated with various materials to impart additional desired properties such as increased or decreased wettability. Both hydrophilic and hydrophobic thin coatings such as polyvinylpyrrolidone and polyvinylidine difluoride, respectively applied by methods including solvent spraying, dip-casting, photochemical coupling, and plasma deposition can aid in imaging in the presence of different liquids.

The endoscope can be used with a standard white light and/or a light source that can help with a visual diagnosis. In one embodiment, the light source can include ultraviolet (UV) wavelength such that a molecule fluoresces or auto-fluoresces. For example, collagen auto-fluoresces between 270-370 nm; therefore a 310 nm UV light source could be utilized to observe collagen in a root canal. A molecular tag can also be used during a clinical procedure, and the appropriate light source can be used to observe the tagged molecule. For example, the molecule fluorescein can when used as a mouthwash will bind to dental caries. An appropriate light source emitting at 494 nm (e.g., 400 nm to 600 nm) attached to the endoscope can be used to produce fluorescent images of visible caries. Other wavelengths of electromagnetic (EM) radiation, such as X-ray and infrared (IR) can be used for imaging through the dental endoscope as well. The endoscope also may contain a detector to measure the amount of fluorescence emitted. The observed images, video or streaming video can be taken under white or UV light, or X-ray or IR EM. IR can be used for a qualitative dental diagnosis. The different illumination energies in one embodiment can be turned on individually and in another embodiment can be turned on together. Fluorescence measured by the detector on the endoscope can provide quantitative information about the clinical issues. Software algorithms can perform analysis and provide information to the clinician such as size measurement, density calculations, porosity calculations, fluorescents intensity and image manipulation.

Visible and fluorescent stains selected based upon their interaction with dental carries and dental root pulp structures can also be used and selected based upon the illumination source. For example, under visible light, Alician Blue, high- or low-iron diamine, aldehyde fuchsin, dialyzed iron ferricyanide, Azure A, and Cuprolinic blue each bind glycosaminoglycans, Alizarin Red S binds calcium, Gomori's trichrome stains collagen green to blue, cytoplasm red, and nuclei gray to black, hematoxylin and eosin stain nuclei blue, proteins pink, and cytoplasm, connective tissue, colloid, and decalcified bone matrix pink to red, Van Gieson, Mallory Trichrome, and Masson Trichrome bind collagen (the latter also stains cytoplasm pink and nuclei black), Oil Red O and Osmium Tetroxide each bind lipids, Von Kossa Stain binds calcium. When used with a polarized light source, PicroSirius Red images collagen.

Appropriate fluorescent nucleic acid stains available from Sigma-Aldrich (St. Louis, Mo.) include: 4-Acetamido-4′-isothiocyanato-2,2′-stilbenedisulfonic acid, Acridine Orange, Acridine Mutagen ICR 191, Acriflavine, Actinomycin D, 6-Aminofluorescein, Albumin blue 580 potassium salt, 4-Aminobenzamidine dihydrochloride, 7-Amino-4-methyl-3-coumarinylacetic acid, 8-Anilino-1-naphthalenesulfonic acid, 9,10-Anthracenediyl-bis(methylene)dimalonic acid, Atto 520, Atto 520 NHS ester, Atto 565, Atto 565 NHS ester, Atto 590, Atto 590 NHS ester, Atto 610, Atto 610-NHS ester, Atto 655, Atto 655 NHS ester, Atto 680, Atto 680 NHS ester, Atto-Dino 2, Atto-Dino 4, Atto-Mono 1, Atto-Mono 2, N-[4-(2-Benzimidazolyl)phenyl]maleimide, BETO, bisBenzimide H 33258, bisBenzimide H 33342 trihydrochloride, [2,6-Bis(2-pyridyl)phenyl-C,N,N′]chloroplatinum(II), 3-Bromomethyl-7-methoxy-1,4-benzoxazin-2-one, 4-Bromomethyl-7-methoxycoumarin, BOXTO, 5-(Bromomethyl)fluorescein, 9-(2-Carboxy-2-cyanovinyl)julolidine, 5-Carboxyfluorescein, 5(6)-Carboxyfluorescein, 5(6)-Carboxyfluorescein diacetate, 5-Carboxyfluorescein N-succinimidyl ester, 5(6)-Carboxyfluorescein N-hydroxysuccinimide ester, 5(6)-Carboxy-X-rhodamine, 5-Carboxy-X-rhodamine N-succinimidyl ester, 5(6)-Carboxy-X-rhodamine N-succinimidyl ester, 5(6)-Carboxytetramethylrhodamine, 6-Carboxytetramethylrhodamine, 5-Carboxy-tetramethylrhodamine N-succinimidyl ester, 5(6)-Carboxytetramethylrhodamine N-succinimidyl ester, 6-Carboxy-tetramethylrhodamine N-succinimidyl ester, 4-Chloro-7-nitrobenzofurazan, Chromeo™ P465, Chromeo™ P503, Chromeo™ P540, 1,3-Cyclohexanedione, Dansyl chloride, Dansylcadaverine, 4′,6-Diamidino-2-phenylindole dihydrochloride, Dimidium bromide, 9-(2,2-Dicyanovinyl)julolidine, Dihydroethidium, Fluorescent red 630, NIR-641 N-succinimidyl ester, NIR-797 isothiocyanate Trioxsalen, 3,5-Diaminobenzoic acid, 3,5-Diaminobenzoic acid dihydrochloride, Eosin Y disodium salt, Ethidium bromide, Ethidium homodimer, Fluram, LUCY® 506, LUCY® 565, LUCY® 569, 6-[Fluorescein-5(6)-carboxamido]hexanoic acid N-hydroxysuccinimide ester, Fluorescein 5(6)-isothiocyanate, Fluram, Fluorenylmethoxycarbonyl chloride, N-(Iodoacetaminoethyl)-1-naphthylamine-5-sulfonic acid, 5-Maleimido-eosin, 2-Methoxy-2,4-diphenyl-3(2H)-furanone, 8-Methoxypsoralen, 4-Methylumbelliferyl-N,N′-diacetyl-β-D-chitobioside monohydrate, Monochlorobimane, Nancy-520, Propidium iodide, Pyrene, Pyronin Y, SYBR® Green I, SYBR® Green II, Streptavidin-CryptoFluor™ Crimson, Sulforhodamine B acid chloride, SYPRO® Orange, SYPRO® Red, SYPRO® Ruby, SYPRO® Tangerine, and Tetramethylrhodamine isothiocyanate.

In another embodiment as shown in FIG. 6, an endoscope assembly 20 may be provided having an integral device 22 (FIG. 6) with a removable (e.g., disposable such as for single use purposes) end portion 24. More particularly, the integral endoscope assembly 22 may include a body portion 26 (which may house camera, battery, and/or handle components) and a fiberscope portion 28 (which may include a flexible and/or straight (rigid) probe 29). Furthermore, the end portion 24 (multiple use purposes) of the assembly 20 may be sterilized in between patients to prevent biocontamination between patients.

In another embodiment as shown in FIG. 5, an endoscope assembly 30 may be provided having various removable components, which include a removable optics head 38 with integral optical probe 39 that can be sterilizable between patients and a body 36. Optionally, the optical head 38 may further include a removable (e.g., disposable sheath) end portion 34 placed over it to prevent biocontamination between patients. Various designs and inventions are covered in a variety of disclosures and patents by Vision Science, see references, which are herein incorporated by reference for all purposes.

As shown in FIGS. 7-10, an alternative embodiment of the removable end component 44 (e.g. protective sheath) is provided. The removable end component 44 may include a connection end 46 having a first end for removable attachment to the fiberscope component and/or the handpiece body and a second end in communication with a tapered guide tube shaft 48 extending therefrom to a scope shaft 50 having a closed (e.g., sealed) tip portion 52. In one specific example, the tip portion 52 includes transparent one or more windows 54 located about the surface perimeter of the tip portion 52 and at a free end 56 of the scope shaft 50 to allow transmission of light therethrough to illuminate the respective cavity (e.g., root canal) while optionally allowing capture of still images and/or video stream of the illuminated cavity. It is appreciated that one or more widows may be located anywhere along the perimeter surface and/or the free end 56 of the scope shaft 50, though not required.

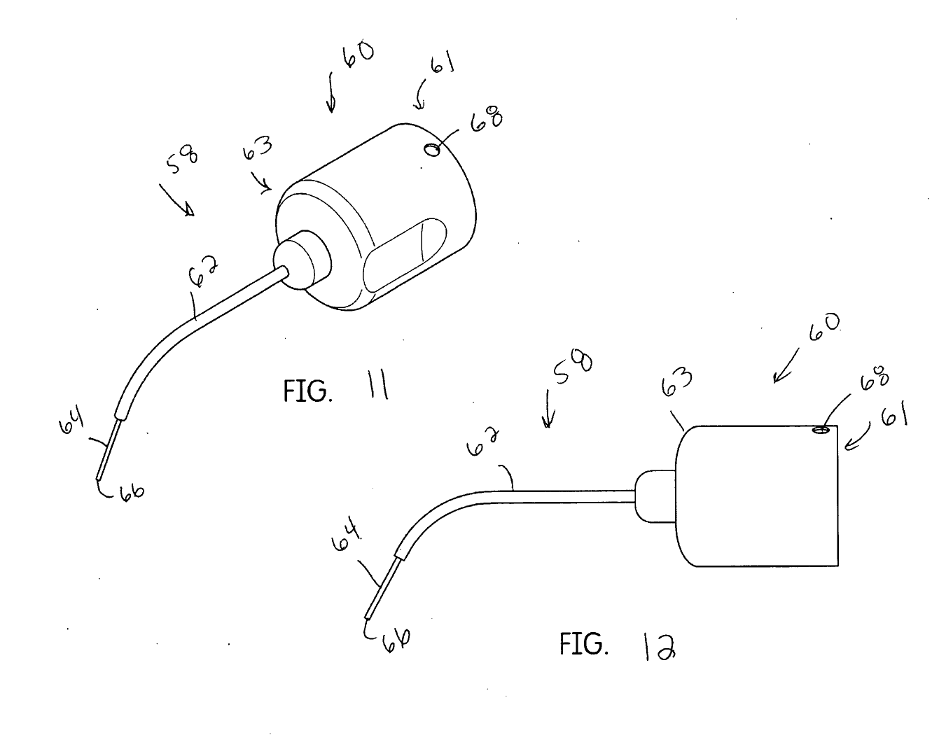

As shown in FIGS. 11-12, an alternative embodiment of the fiberscope component (e.g., probe) 58 is provided. The fiberscope component 58 may include a connection end 60 having a first end 61 for removable (or non-removable) attachment to the handpiece body and a second end 63 in communication with a guide tube shaft 62 extending therefrom to an optical shaft 64 having a free end 66, which defines a hollow pathway for providing illumination and/or transmitting (sending and/or receiving) data such as still images, video streaming, material transport, or otherwise. It is appreciated that connection end may be a quick-disconnect and/or include locking component 68 to releasably/removably secure the fiberscope component 58 to the handpiece body, though not required. When included, the handpiece body may further include a mating component (not shown), which corresponds to locking portion 68 for removeable securement of the fiberscope component 58 to the handpiece body.

Various locking/removable securement embodiments known in the art are contemplated as viable alternatives. Example of quick disconnect connectors include, but are not limited to those commonly used in the fiber optic industry today, as well as those used in other industries whose designs are amenable to fiber optic cables. These include detachable rigid and semi-rigid probes (Zibra Corp, Westport Mass.), multichannel fiber optic connectors as described in U.S. Pat. No. 4,432,603, hermaphroditic fiber optic connectors (Optical Cable Corporation, Roanoke, Va.), Quick LC fiber optic connectors (Extron Electronics, Anaheim, Calif.), NSK quick connect adapters (Dental Parts Haus, Richmond, Va.), FuseConnect fusion spliced field installable connectors (AFL, Duncan, S.C.), E-2000 connectors (Senko, Dallas Tex.), and other Lucent Connectors (LC), Snap-in connectors (SC), Straight Tip Connectors (ST), Ferrule Connectors (FC), Mechanical Transfer Registered Jack Connectors (MT-RJ), Miniature Unit Connectors (MU), “Subminiature A” connectors (SMA), and DIN connectors. Designs that could readily be adapted to fiber optics include the quick change collet nuts (Dremel, Mount Prospect, Ill.), hexagonal ended drill bits (Black and Decker, New Britain, Conn.), push connectors (e.g., Legris D.O.T. Push-In Union Elbow, Dixon Valve, Chestertown, Md.), Swagelok fittings (Swagelok, Solon, Ohio), other industrial couplings including CEJN plug, Hansen Plug, Sharader Plug, Chicago Coupling, Snap-Tite Coupling, JIFFY-TITE™ quick-connects, Barbed Leur-lock connectors, OHMEDA STYLE quick connects, snap closures, military closures, garden hose quick connect fittings and couplings, propane gas quick connect fittings, fire-hose closures.

It is further contemplated that the clinician may inject the dyes, stains, and fluorophores into the patients root canal or other intra-oral areas with a standard syringe prior to inserting the mini-endoscope probe for imaging. Other methods include using a lumen built into the disposable sheath, see U.S. Pat. No. 5,025,778, to inject the fluid while the mini-endoscope probe is in the canal or mouth. Per the referenced patent, this open lumen can also be a means to dry the tooth canal using vacuum without removing the optical probe when a dry canal provides better environment for the image acquisition.

Software algorithms are able to convert the digital images of fluorescing molecules to maps or counts to determine the level of canal cleaning that exists. The invention could also display this information as a qualitative indicator of cleaning or can be a quantitative score presented on the heads-up display or monitor.

Optical imaging using optical coherence tomography (OCT) can be used in acquiring 3D information in the root canal of the tooth that will help the dentist in diagnosis or treatment. The small fiber size used in this invention is capable of being placed in the canal and support the short wavelengths needed for low-coherence interferometry. The optical head and fiber optic tip can be modified to include a fiber-optic splitter to create the sample reference arms of the interferometer for the data stream. OCT analysis algorithms of the data can display the results in the heads-up display or on a monitor. These OCT probes can also be used outside the tooth intra-orally.

Furthermore, the present invention may include one or more of the following features:

The handpiece or device may include a mini endoscope for inspecting the tooth root canal has been described in the literature, for examples see CN2617308Y Digital optical fibre root canal microendoscope device, Development of a New Micro-Endoscope for Odontological by Geibel Kopie, AO_{—}104_{—}1-2_Drs_Moshonov_and_Nahlieli_{—}2_, and several Vision Science patents for protective disposable sheathes.

The handpiece or device may contain a visible light Illumination source and the light is transmitted in an outer annulus around the image fiber to the distal end of the tip to illuminate the canal.

The Outer Diameter of optical fiber head may range from 0.25 mm to 1.0 mm for in the canal. Up to 10 mm intra oral applications.

The Outer Diameter of the protective sheath/guide tube may range down to 0.35 mm.

Wavelength of illumination may range from 270-370 mm for uv and/or at least include all uv spectrum due to various fluorophores.

All visible IR to at least 3000 nm.

The image fiber may include 0.35-1.0 mm outer diameter and has 3,000-10,000 pixels of information for capture by a CMOS or CCD digital camera.

The optical fiber has a flexible portion of the fiber ˜8-15 mm in length that protrudes beyond a protective tube and can be inserted into a portion of the body, in this case inside the root canal or other intra-oral portions of the mouth.

The Depth of Field of the fiber optic is up to 10 mm.

A mirror or lens can be located at the distal end of the fiber or the fiber end can be angled to allow side viewing as well as axial viewing.

The handpiece or computer contains lensing and a CMOS or CCD digital camera for capturing the image fiber output and a microprocessor and algorithms for displaying the still or video images onto a computer screen or monitor.

The computer can display the images with digital magnification.

The video or still images can be stored on a computer system.

The fiberoptic tip can be re-usable (re-sterilizable) or protected from bio-contamination by a disposable protective sheath with an optical window at the distal end.

The invention described herein has many other advantages. The endodontic instrument may have a single continuous flow path, which eliminates potential leak paths. Inherent stress concentrations may be reduced or substantially eliminated, thereby allowing the tip and/or the distal end portions to be reliable during vibration. The configuration of the tip and/or the distal end portions guide and transfer the ultrasonic vibration and energy in the planes of motion, which provides proper agitation to the irrigants. The tip assembly can also be disposable, thereby requiring that a new tip assembly be used for each patient and insuring that the tip assembly will be sterile prior to use.

The handpiece may contain the electronics, wireless communications, software, algorithms, batteries, and imaging camera. The nosecone contains the fiberscope, lens, and optical image fiber. There may or may not be a disposable guide tube that slides over the imagefiber to protect it from damage or bio-contamination. FIG. 4 shows the unit in a clinician's hand and FIG. 5 is a representation of the device in use and the image being displayed to a monitor.

Each feature disclosed in this specification (including any accompanying claims, abstract, and drawings), may be replaced by alternative features having the same, equivalent or similar purpose, unless expressly stated otherwise. Thus, unless expressly stated otherwise, each feature disclosed is one example only of a generic series of equivalent or similar features.

While preferred embodiments of the present invention have been shown and described herein, it will be obvious to those skilled in the art that such embodiments are provided by way of example only. Numerous variations, changes, and substitutions will be apparent to those skilled in the art without departing from the invention. Other foreseen embodiments or uses for the present invention include the use of the invention in the field of phacoemulsification, where a tip assembly such as the present invention may offer many advantages. Accordingly, it is intended that the invention be limited only by the scope of the appended claims.

1. A system for acquiring and sharing in real time video and/or still images with a tooth or tooth root during a dental procedure comprising an endoscope.

2. The system of claim 1 further comprising a heads up display in communication with the endoscope for displaying in real-time the video and/or still images

3. The system of claim 1, wherein the endoscope includes a fiber optic material.

4. The system of claim 3, wherein the fiber optic material is a quartz fiber so that other wavelengths including UV, IR, or a combination of both for excitation.

5. The system of claim 1, wherein the endoscope further includes a removable sheath having a hydrophilic or hydrophobic exterior coating.

6. The system of claim 2, wherein the endoscope employs an OCT design and software algorithms to image the root canal walls.

7. The system of claim 1 further comprising a micro-fiberscope, an illumination source, a camera, a wired or wireless transmission system, and a display.

8. The system of claim 7, wherein the display is a television, tablet, smartphone or computer monitor.

9. The system of claim 8, wherein the display is a heads-up display.

10. The system of claim 8, wherein the heads-up display is Google Glass.

11. The system of claim 8, wherein the heads-up display is Samsung Galaxy Glass.

12. The system of claim 3, wherein the fiber optic material is selected from the group consisting of polymeric optic fibers, glass-ceramic optic fibers, and quartz optic fibers.

13. The system of claim 1, wherein the endoscope includes a fiberscope having a dental probe detachable from a main fiber optic cable.

14. The system of claim 13, wherein the probe attaches to the main fiberoptic cable through a quick connector.

15. The system of claim 13, wherein the probe is constructed of all polymeric materials.

16. The system of claim 13, wherein the probe is resterilizable.

17. The system of claim 13, wherein the probe is disposable

18. The system of claim 13, wherein the probe includes a probe tip that is modified or coated to make it hydrophilic.

19. The system of claim 13, wherein the probe includes a probe tip that is modified or coated to make it hydrophobic.

20. The system of claim 13, wherein the endoscope further includes a disposable sheath, which protects the probe from being biocontaminated.

21. A method for acquiring and sharing in real time video and/or still images with a tooth or tooth root during a dental procedure comprising the step of illuminating a cavity of a root canal and acquiring still images and or video of the illuminated cavity of the root canal.

22. The method of claim 21 further comprising the step of applying fluorophores and/or dyes to a cavity with a root of a tooth to tag and/or stain bacteria, molds, and/or other molecules so they will fluoresce under UV, IR or other wavelengths for identification by the imaging system.

23. Method of claim 22 wherein the applying fluorophores and/or dyes step includes using UV 270-370 nm to fluoresce collagen in the root canal.

24. Method of claim 21 wherein the illumination step includes using blue visible light to fluorescence dentin in the root canal.

25. The method of claim 21, wherein the video and/or still images are acquired using a micro-fiberscope, illumination source, and camera.

26. The method of claim 22, wherein the video and/or still images are wirelessly transmitted to one or more heads-up displays.

27. The method of claim 22, wherein the video and/or still images are transmitted via a wired connection to one or more heads-up displays or network with one or more monitors or heads-up displays.

28. The method of claim 22, wherein the video and/or still images are transmitted over a wireless network to one or more heads-up displays.

29. The method of claim 21 further comprising the step of transmitting images and/or videos with a patient, clinical staff, consultants for patient education, dental staff training, consultant referrals or consultations, or any combination thereof.

30. The method of claim 21, wherein the video and/or still images of a feature or structure are acquired using a micro-fiberscope, fluorophore, illumination source of wavelength capable of effecting fluorescence of the fluorophore, and a camera.

31. The method of claim 22, wherein the fluorophore is present naturally within the tooth.

32. The method of claim 21, wherein the illumination source emits UV light of 270-370 nm wavelength.

33. The method of claim 30, wherein the structure is collagen.

34. The method of claim 22, wherein the fluorophore is sodium fluorescein.

35. The method of claim 22, wherein the fluorophore is applied to the tooth after endodontic preparation from a solution.

36. The method of claim 21, wherein the illumination source emits visible light.

37. The method of claim 21, wherein the video and/or still images are transmitted to a display.

38. The method of claim 37, wherein the display is selected from the group consisting of a television, a computer monitor, and a heads-up display.

39. The system of claim 3, wherein the microfiber is 0.01 mm or larger.

40. The system of claim 39 wherein the microfiber is between 0.1 mm and 10 mm.

41. The method of claim 29 wherein the transmission is via a wireless signal.
